(19) United States
(12) Patent Application Publication
(10) Pub. No.: US 2010/0049282 A1
(43) Pub. Date: Feb. 25, 2010

(54) Phototherapy Apparatus for Hair Loss Treatment

(75) Inventor: Sean Xiaolu Wang, Wilmington, DE (US)

Correspondence Address: BWT PROPERTY, INC., 19 SHEA WAY, SUITE 301, NEWARK, DE, 19713, US

(73) Assignee: BWT PROPERTY, INC., Newark, DE (US)

(21) Appl. No.: 12/544,278

(22) Filed: Aug. 20, 2009

Related U.S. Application Data
(60) Provisional application No. 61/090,925, filed on Aug. 22, 2008.

(51) Int. Cl. A61N 5/067

(52) U.S. Cl. 607/89

(57) ABSTRACT

A phototherapy apparatus for hair loss treatment. The phototherapy apparatus comprises a laser array to produce a plurality of laser beams and a beam shaping component to control the divergence angle and intensity distribution of the laser beams so as to produce a desired light intensity distribution on the surface of the scalp tissue to be treated.

### REFERENCE TO RELATE APPLICATION

This application claims an invention which was disclosed in Provisional Patent Application No. 61/090,925, filed Aug. 22, 2008, entitled “PHOTOTHERAPY APPARATUS FOR HAIR LOSS TREATMENT”. The benefit under 35 USC §119(e) of the above mentioned United States Provisional Applications is hereby claimed, and the aforementioned application is hereby incorporated herein by reference.

### FIELD OF THE INVENTION

This invention generally relates to a phototherapy apparatus, and more specifically to a phototherapy apparatus for hair loss treatment.

### BACKGROUND

Low light laser therapy (LLLT) can be applied to the scalp for hair loss treatment. The beneficial effects of this photo-biostimulation process include increase in ATP and keratin production, enhancement in blood flow and circulation, as well as increase in the number of red corpuscles. The scalp has a relatively large surface area that can not be covered with one single laser beam. Thus the previous disclosed LLLT device either uses a large number of laser beams (U.S. Pat. No. 7,201,764, European patent No. EP-0130950) or uses a limited number of laser beams and scans the laser beams across the scalp (U.S. Pat. Nos. 6,666,878 and 6,936,044). The present invention discloses a new LLLT device that offers better uniformity in light intensity and enhanced penetration depth control over those previous approaches.

### SUMMARY OF THE INVENTION

A phototherapy apparatus for treatment of a skin tissue, the phototherapy apparatus comprising: a plurality of lasers to produce a plurality of laser beams; and at least one beam shaping component associated with the plurality of laser beams for controlling the divergence angle and intensity distribution of the laser beams and producing a plurality of transformed laser beams; wherein the transformed laser beams mix on a surface of the skin tissue to produce a predetermined intensity distribution thereof.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates the first exemplary embodiment of the present invention.

FIG. 2 illustrates the second exemplary embodiment of the present invention.

FIG. 3 illustrates the third exemplary embodiment of the present invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a phototherapy apparatus for hair loss treatment. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

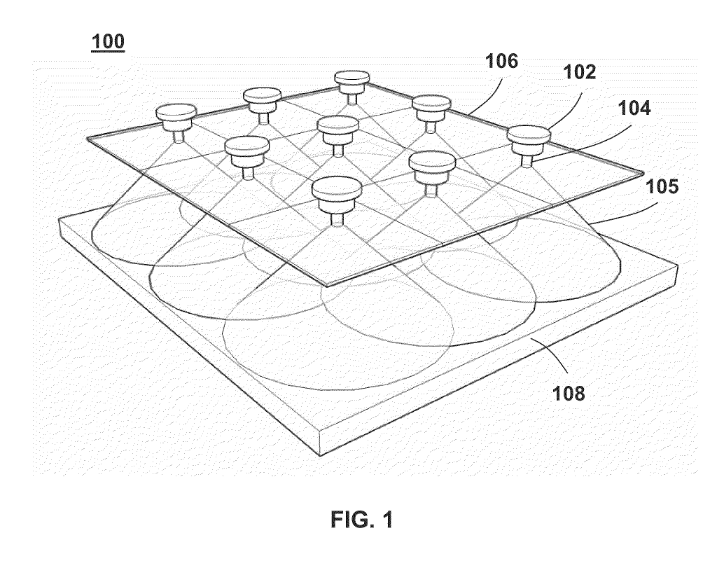

In the first exemplary embodiment of the present invention as shown in FIG. 1, the phototherapy apparatus 100 comprises an array of laser diodes 102, each producing a laser beam 104. The wavelength of the laser beam may range from visible to near infrared with a preferable value at around 670 nm. The phototherapy apparatus 100 further comprises a beam shaping component 106, preferably in the form of a holographic optical diffuser, which expands the divergence angle of the laser beam 104 and in the meantime homogenizes the beam's intensity distribution. The transformed laser beams 105 overlap on the surface of the subject scalp 108 to produce a uniform illumination. The divergence angle of the transformed laser beams 105 is accurately controlled by the view angle of the holographic diffuser in a manner that θₒ²=θᵢ²+θ_{d}², where θₒ is the divergence angle of the transformed laser beam 105, θᵢ is the divergence angle of the input laser beam 104, and θ_{d} is determined by the view angle of the holographic optical diffuser. Thus the light intensity on the surface of the scalp 108 is well defined within a predetermined range. By operating the laser diodes 102 in a pulsed mode, a pulsation stimulation of the scalp can be produced.

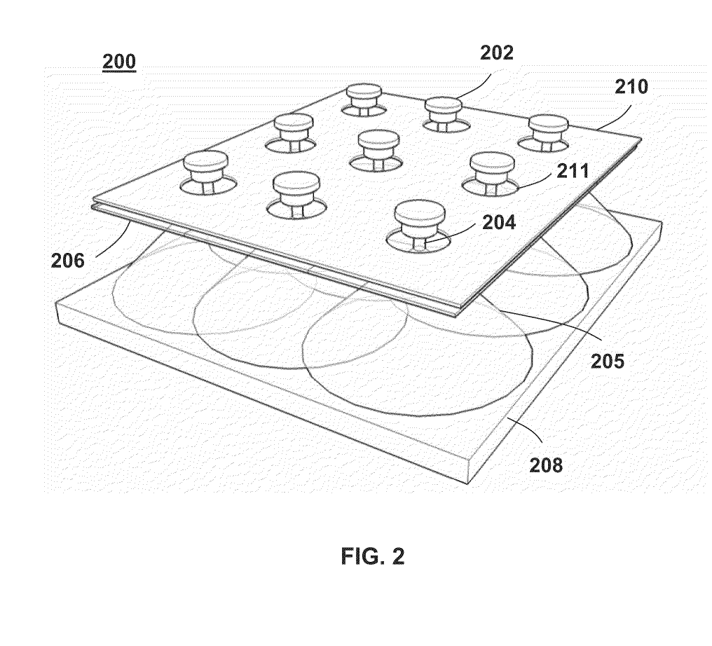

In the second exemplary embodiment of the present invention as shown in FIG. 2, the phototherapy apparatus 200 comprises an array of laser diodes 202, a beam blocker 210, and a beam shaping component 206. The beam blocker 210 comprises an array of holes 211 matched with the laser beams 204 that are produced by the laser diodes 202. The beam shaping component 206 works similarly as does in the first embodiment to control the divergence angle and intensity distribution of the laser beams 204. By controlling the relative position of the laser diodes 202 and the beam blocker 210, the laser beams 204 either pass through the holes 211 or be blocked out, producing a pulsation stimulation effect to the subject scalp 208.

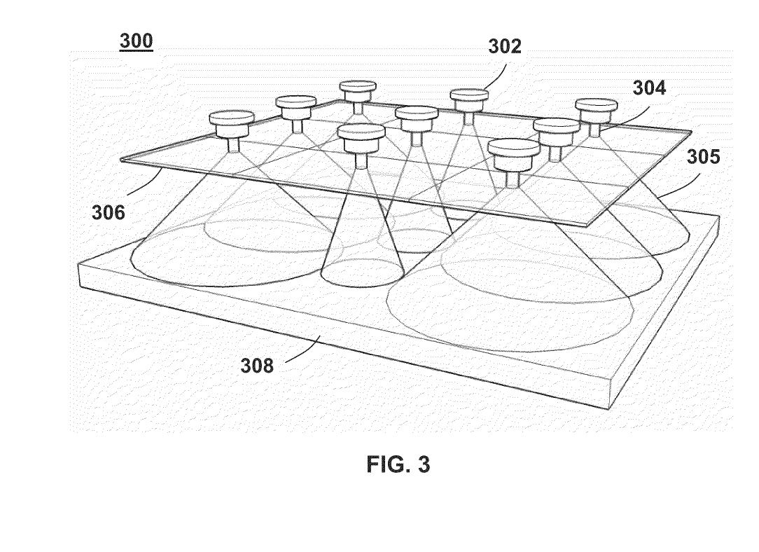

In the third exemplary embodiment of the present invention as shown in FIG. 3, the phototherapy apparatus 300 comprises an array of laser diodes 302 and a non-uniform beam shaping component 306. The beam shaping component 306 can be a non-uniform holographic optical diffuser with multiple sections. Different sections possess different view angles, thus making the transformed laser beam 305 obtain different divergence angles. The laser beam with smaller divergence angle produces higher light intensity and penetrates deeper into the scalp 308 while the laser beam with larger divergence angle produces lower light intensity and smaller penetration depth. By scanning the non-uniform beam shaping component 306 across the surface of the scalp 308, both the inner layer and outer layer of the scalp tissue can be effectively treated.

In all the above embodiments, the laser diodes may have different output wavelengths to provide different penetration depth and photo-biostimulation effect. For example, laser diodes with 670 nm output wavelength will produce a penetration depth of 8-10 mm for direct treatment of hair organ. In comparison, laser diodes with longer output wavelengths, such as 830 nm, will produce a penetration depth of 15-20 mm for increasing the micro-circulation of inner scalp tissue. By moving the laser diode array across the scalp surface, the scalp tissue can be consecutively treated with laser beams of different wavelengths.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A phototherapy apparatus for treatment of a skin tissue, the phototherapy apparatus comprising:
a plurality of lasers to produce a plurality of laser beams; and
at least one beam shaping component associated with the plurality of laser beams for controlling the divergence angle and intensity distribution of the laser beams and producing a plurality of transformed laser beams;
wherein the transformed laser beams mix on a surface of the skin tissue to produce a predetermined intensity distribution thereof.

2. The phototherapy apparatus of claim 1, wherein the lasers operate in a pulsed mode.

3. The phototherapy apparatus of claim 1, wherein the lasers operate in a continuous mode.

4. The phototherapy apparatus of claim 1, wherein the beam shaping component controls the plurality of transformed laser beams to have different divergence angles.

5. The phototherapy apparatus of claim 1, wherein the plurality of lasers and the beam shaping component is movable over the surface of the skin tissue.

6. The phototherapy apparatus of claim 1, further comprising a beam blocker between the plurality of lasers and the beam shaping component, wherein the beam blocker is movable over the surface of the skin tissue.

7. The phototherapy apparatus of claim 1, wherein the beam shaping component comprises a holographic optical diffuser.

8. The phototherapy apparatus of claim 1, wherein the plurality of lasers comprise diode lasers.

9. The phototherapy apparatus of claim 1, wherein the plurality of lasers comprises lasers of different output wavelengths.
